(19) United States
(12) Patent Application Publication
(10) Pub. No.: US 2020/0342957 A1
(43) Pub. Date: Oct. 29, 2020

(54) CHEMICAL BINDING SIMILARITY SEARCHING METHOD USING EVOLUTIONARY INFORMATION OF PROTEIN

(71) Applicant: KOREA INSTITUTE OF SCIENCE AND TECHNOLOGY, Seoul (KR)

(72) Inventors: Keunwan PARK, Gangneung-si (KR); Cheol-Ho PAN, Gangneung-si (KR); Young-Joon KO, Gangneung-si (KR); Prasannavenkatesh DURAI, Gangneung-si (KR); Yongsoo CHOI, Gangneung-si (KR); Moon-Hyeong SEO, Gangneung-si, Gangwon-do (KR); Kyungsu KANG, Gangneung-si (KR); Jin Soo PARK, Gangneung-si (KR); Jaeyoung KWON, Gangneung-si (KR)

(21) Appl. No.: 16/857,765

(22) Filed: Apr. 24, 2020

(30) Foreign Application Priority Data
Apr. 25, 2019 (KR) 10-2019-0048391

(51) Int. Cl. G16B 20/30; G16B 40/20; G16B 15/30

(52) U.S. Cl. CPC G16B 20/30; G16B 15/30; G16B 40/20

(57) ABSTRACT

The present invention relates to an ensemble evolutionary chemical binding similarity (ensECBS) model, which is a chemical binding similarity searching method widely applicable as a powerful tool for representing an unknown relationship between chemicals by using evolutionary information of proteins binding to chemicals.

### BACKGROUND OF THE INVENTION

### 1. Field of the Invention

The present invention relates to an ensemble evolutionary chemical binding similarity (ensECBS) model, which is a universally applicable chemical binding similarity searching method as a powerful tool for representing a functional relationship related to chemical-target binding by using evolutionary information of proteins binding to chemicals.

### 2. Description of the Related Art

A chemical similarity searching technique is a common tool widely used as a method of searching for similar chemicals from chemical databases. Most similarity searching methods, however, focus on measuring the overall structure similarity of chemicals. Therefore, there is a limit to representing protein binding of chemicals or functional similarity of chemicals, which is often caused by local pharmacophore features.

A representative method of calculating the structure similarity of chemicals is a method of calculating the Tanimoto coefficient using chemical fingerprint vectors. The chemical fingerprint vector is to represent a chemical in the form of a vector in which local fragments frequently found in the chemical are predefined and a digit of 0 or 1 is assigned depending on the presence or absence of a specific local fragment. The chemical fingerprint vector may have different sizes and values depending on how the local fragments of the chemical are collected. As the fingerprint vector, a variety of fingerprints such as PubChem, FPset, Atom Pair, MACCS fingerprint, etc. are used (https://openbabel.org/wiki/Tutorial: Fingerprints, https://www.bioconductor.org/packages/devel/bioc/vignettes/ChemrmnneR/inst/dc/ChetmmineR.html#fpfpset-classes-for-storing-fingerprints).

The structure similarity of the chemicals can be calculated by comparing the chemical fingerprint vectors with each other, and the structure similarity is mainly calculated through the Tanimoto coefficient method. The Tanimoto coefficient is a ratio of the number of common local fragments to the total number of local fragments found in the chemical fingerprint vector, and has a value between 0 and 1. The Tanimoto coefficient closer to 1 indicates that two chemicals are structurally more similar (Bajusz, D., Racz, A. and Heberger, K. (2015) Why is Tanimoto index an appropriate choice for fingerprint-based similarity calculations?J Cheminformatics, 7:20).

The searching method of using the chemical fingerprint vector and Tanimoto coefficient is the most widely used chemical similarity searching method, and has an advantage in that searching is rapid and its application is easy. However, since the method calculates the score of the overall structural similarity, there is a great limitation in representing sensitive local pharmacophore features related to the target binding or function of the chemical, and there is a disadvantage in that predictability of functionality is also considerably reduced.

The chemical fingerprint vector varies in the value depending on how the chemical local fragments are defined, and mostly the secondary chemical structure (atomic and binding linkage information) of the chemical is considered, and therefore, for the improvement thereof, a three-dimensional chemical shape similarity searching method has been developed. This method has an advantage of better representation of three-dimensional structural features of chemicals than the method of using chemical fingerprint vectors. However, the chemical shape similarity searching method also focuses on representing the overall structure similarity rather than representing information of functionally important features of chemicals (https://github.com/ambrishroy/LiGSIFT, http://insilab.org/lisica/).

Therefore, there is a need to develop a new method capable of determining protein binding of chemicals or functional similarity of chemicals, which is caused by local features.

Meanwhile, binding of chemical compounds to the target proteins is the most important information in revealing the mechanism of action and function of the chemical. However, the chemical-target binding is associated with the complex three-dimensional molecular structural features, and thus there are many limitations in representing it through the above-mentioned general-purpose chemical structure similarity searching methods. For this reason, research is mainly conducted through a nonlinear computational model. Typically, quantitative structure activity relationship (QSAR) studies are actively conducted through machine learning methods (Luo, M., Wang, X. S. and Tropsha, A. (2016) Comparative Analysis of QSAR-based vs. Chemical Similarity Based Predictors of GPCRs Binding Affinity. Mol Inform, 35, 36-41).

The QSAR model refers to a predictive model generated by applying a statistical correlation between structural features and biological activity of a chemical using a molecular descriptor representing a molecular structure or feature. In particular, the activity to be predicted may include various characteristics such as inhibition of target function, exploration of new drug candidates, lead optimization, risk assessment, toxicity, etc.

However, since QSAR research considers the complex molecular features associated with the pre-defined target proteins, it is not generally applicable to various targets, and it is not applicable when there is no information about a chemical binding to a specific target. Therefore, despite the high predictability of the machine learning-based QSAR study, it is difficult to generally represent the similarity relationship between chemicals, such as the chemical binding similarity proposed in the present invention.

Accordingly, there is a demand for a chemical similarity searching technique that is generally applicable and capable of representing target binding similarities for better representation of the functional relationship of chemicals.

Under the circumstances, the present inventors have made intensive efforts to develop a chemical similarity searching technique, which is useful to search for chemicals having similar functions and to reveal the mechanism of action of chemicals, and as a result, they have developed an ensemble evolutionary chemical binding similarity (ensECBS) model which is a general-purpose chemical binding similarity searching method using chemical-target binding information and targets' evolutionary information. It was found that this developed method is effective for finding hidden chemical binding similarities even with insufficient data of chemicals which are known to bind to targets, and serve as a novel chemical similarity searching tool that uses evolutionarily conserved target binding information, thereby completing the present invention.

### SUMMARY OF THE INVENTION

An object of the present invention is to provide a chemical binding similarity searching method using evolutionary information of proteins binding to chemical compounds.

### BRIEF DESCRIPTION OF THE DRAWINGS

FIG. 1 shows a schematic illustration of categorization of chemical pairs by considering the relationship between chemical, target, and evolutionary information to define chemical binding similarity.

FIG. 2 shows a schematic illustration of a procedure of integrating diverse evolutionary information to develop the chemical binding similarity searching method.

FIG. 3 shows a performance test for prediction of chemical pairs binding to identical targets, as compared with the existing structure similarity method.

FIG. 4 shows prediction accuracy of drug pairs binding to Ephrin type-B receptor 4, as compared with the existing 2D structure similarity method.

### DETAILED DESCRIPTION OF THE PREFERRED EMBODIMENTS

Hereinafter, the present invention will be described in detail. Meanwhile, each description and embodiment disclosed in this disclosure may also be applied to other descriptions and embodiments. That is, all combinations of various elements disclosed in this disclosure fall within the scope of the present invention. Further, the scope of the present invention is not limited by the specific description described below.

Further, those skilled in the art will recognize or be able to ascertain, using no more than routine experimentation, many equivalents to the specific embodiments of the invention described herein. Further, these equivalents should be interpreted to fall within the present invention.

To achieve the object, an aspect of the present invention provides a chemical binding similarity searching method using evolutionary information of proteins binding to chemical compounds.

The chemical binding similarity searching method of the present invention is a calculation method capable of determining chemical binding similarity from structural information of chemical compounds, and is a machine learning model-based chemical similarity searching method of comprehensively using structural information of chemicals, chemical-target binding information, and evolutionary information of chemical-binding target.

The chemical binding similarity searching method of using protein evolutionary information of the present invention may include the steps of obtaining chemical-target protein binding information from experimental data; constructing expanded chemical-protein interaction data by using diverse evolutionary information of the target proteins; categorizing the interaction data into positive and negative chemical pairs and quantitating the data; and applying a machine learning-based classification model to the quantitated data to calculate a chemical binding similarity score.

The chemical-protein “binding information” of the present invention may be defined by physical molecular binding, and binding affinity may be represented by values such as Ki (inhibition constant), IC50, Kd, EC50, etc.

Comprehensive chemical-protein interaction data may be constructed by collecting the chemical-protein binding information from the database. Information about chemical-protein pairs having binding affinity of a specific value or higher, based on the binding affinity criteria, may be collected and used as chemical-protein binding information data.

The database for collecting the chemical-protein binding information of the present invention may be DrugBank or BindingDB database, but is not limited thereto.

The “DrugBank” database is a comprehensive on-line database (www.drugbank.ca) containing information on drugs and drug targets. As bioinformatics and cheminformatics resources, DrugBank combines chemical and drug data with comprehensive information of sequence, structure, and pathway thereof.

The “BindingDB” database is an on-line database (www.bndingdb.org) of binding affinity, and contains information focusing chiefly on the interactions between protein targets and small molecules. BindingDB contains about 1,200,000 binding data, for 5,500 protein targets and 520,000 small molecules.

The “evolutionary information” of the present invention is defined through information at multiple levels such as a motif, domain, family, and superfamily. Proteins having the identical evolutionary information are defined as “evolutionarily related proteins”.

In the present invention, data for evolutionary relationship of target proteins may be constructed through motif, domain, family, or superfamily.

The “motif” of the present invention refers to a sequence or a secondary structure when the secondary structure formed by a specific amino acid sequence is found in several proteins.

The “domain” of the present invention refers to a region having a biological function. The domain may consist of several motifs.

The “family” of the present invention means a collection of proteins that are evolutionarily related to each other.

Similarities of amino acid sequences and three-dimensional structures are related to each other. Therefore, as proteins are more closely related, the amino acid sequences and the three-dimensional structures may have higher similarity.

The “superfamily” refers to a relationship established between two families, when identity between family proteins, in which proteins have an amino acid sequence identity of 50% or more, is 30% to 40%.

Interaction data between the chemical and target protein is constructed, and then evolutionarily related chemical pairs are defined and scored by a machine learning model. The output scores for the chemical pairs are applied again to the secondary machine learning-based classification model, and chemical binding similarity may be calculated by the resulting value of the secondary model.

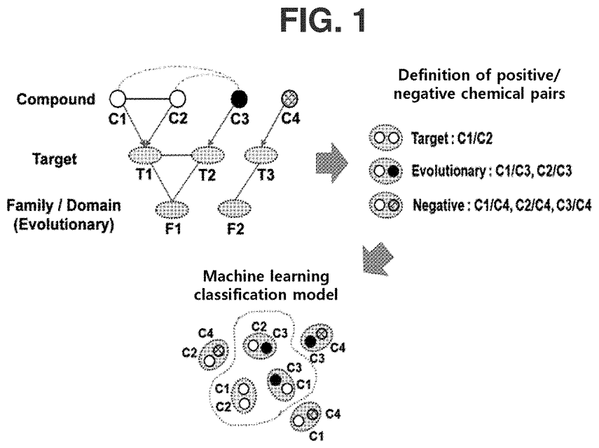

The “evolutionarily related chemical pair” of the present invention is defined by a chemical pair that binds to the same target or to a protein that has the same evolutionary information even if it is not the same target protein. The chemical pair data are categorized into positive chemical pairs and negative chemical pairs to be applied to the machine learning-based classification model. The “positive chemical pairs” are evolutionarily related chemical pairs, and the “negative chemical pairs” may be randomly selected from chemical pairs which are structurally similar to the positive chemical pairs but have no evolutionarily-related target protein (FIG. 1).

In the present invention, the chemical pair data may be numerically represented by using the structural fingerprints of the individual chemicals. The chemical pairs may be expressed by the following equation:

Vij=Vji=Vi+Vi

(V: fingerprint vector, Vi: fingerprint for chemical i, Vj: fingerprint for chemical j)

The “fingerprint vector” of the present invention is a chemical representation where features regarding local fragments frequently found within a chemical are predefined, and ‘absence’ or ‘presence’ of a specific fragment is indicated by 0 or 1. The fingerprint vector may have different size and value depending on how the local fragments of the chemical are collected.

Chemical structure similarity may be calculated by comparing the fingerprint vectors with each other, and structure similarity may be calculated by the Tanimoto coefficient method. The Tanimoto coefficient is a ratio of the number of common local fragments to the total number of local fragments found in the chemical fingerprint vector, and has a value between 0 and 1. The Tanimoto coefficient closer to 1 indicates that two chemicals are more similar in the structure.

Vij which is a fingerprint vector for a chemical pair is represented by the sum of Vi which is a fingerprint vector for arbitrary chemical i and Vj which is a fingerprint vector for chemical j. Therefore, Vij is composed of 0, 1, and 2, where 0 indicates a structural feature present in none of Vi and Vj, 1 indicates a feature present in any one compound of Vi and Vj, and 2 indicates a common feature present in Vi and Vj.

The positive chemical pairs having common evolutionary target information are defined at a motif, domain, family, or superfamily level according to diverse evolutionary information of target proteins, and evolutionary information of each protein may be extracted from diverse protein evolutionary information databases including PFAM, SUPERFAMILY, PRINT, CDD, SMART, G3DSA, INTERPRO, or TIGR.

As used herein, the terms “motif”, “domain”, “family”, and “superfamily” are the same as described above.

The “PFAM” is a database (pfam.xfam.org) for multiple sequence alignments of protein families by a Hidden Markov model. The “SUPERFAMILY” is a database (superfam.org containing structural and functional information for all proteins and genomes. The “PRINT” is a database (130.88.9.239/PRINTS) containing fingerprint information, and may be used as a tool for providing annotation of protein families and determining new sequences. The “CDD” is a database (www.ncbi.nlm.nih.gov) containing functional classification of proteins via superfamily. The “SMART” is a database (smart.emb-heidelberg.de) which may be used for identifying and analyzing protein domains, together with protein sequences. The “G3DSA” is a database (www.ebi.ac.uk/interpro/member-database/CATH-Gene3D) containing functional domain annotations of regulatory proteins. The “INTERPRO” is a database (www.ebi.ac.uk/interpro) of domains, sites in functional proteins, and protein families, in which known proteins can be applied to new protein sequences. The “TIGR” is a database (www.hsls.pitt.edu) containing DNA and protein sequences, gene expression, cellular role, and protein family.

A machine learning classification model for chemical pair data is generated for each evolutionary information of target proteins in particular, applicable machine learning methods may include various methods such as naive bayes classifier, support vector machine, random forest, neural network, and deep learning.

The “naive bayes classifier” is a model trained using not a single algorithm but a family of algorithms based on a common principle, and assumes that all values of particular features are independent of each other. The “support vector machine” is a supervised learning model that recognizes patterns and analyzes data, and is a machine learning-based model used for classification and regression analysis. The “random forest” is an ensemble learning method for classification and regression analysis, and operates by outputting the class or mean prediction from multiple decision trees constructed at training time. The “neural network” is a statistical learning algorithm in machine learning and cognitive science, inspired by biological neural networks. Artificial neural network refers to an entire model that has artificial neurons that form a network of synapses by changing the binding strength of synapses through learning. The “deep learning” refers to the task of extracting the core content or function from high-level abstractions, i.e., large volumes of data or complex data, through a combination of multiple nonlinear transformations.

Diverse definition of the positive chemical pairs of the present invention is possible according to levels of evolutionary information of proteins, and thus multiple positive chemical pair data may be generated. The negative chemical pairs of the present invention are specifically generated for each positive chemical pair data. From multiple definition of positive chemical pairs by different evolutionary information, multiple classification models may be constructed, and each machine learning-based classification model may generate binding similarity scores for chemical pairs in different evolutionary perspective.

The value calculated by the classification model for chemical pairs is a probability value. The value is close to 1 if the feature is similar to the learned positive chemical pairs, and the value is close to 0 if the feature is close to the learned negative chemical pairs. In other words, the value closer to 1 indicates higher probability of binding to an identical target or a protein having the identical evolutionary information according to the kind of the evolutionary information which is used to define the positive chemical pair.

In the construction of ensemble classification model of the present invention, a population of machine learning classification models based on various evolutionary information is constructed, and then the output scores of the machine learning classification model are used to construct the secondary ensemble classification model and to finally obtain the binding similarity scores of the chemical pairs.

An “ensemble evolutionary chemical binding similarity (ensECBS) model.” has been developed, in which chemicals binding to identical targets are finally predicted by combining various classification models generated according to protein target information and evolutionary information. The ensemble evolutionary chemical binding similarity model lastly calculates the probability of arbitrary chemical pairs binding to identical targets.

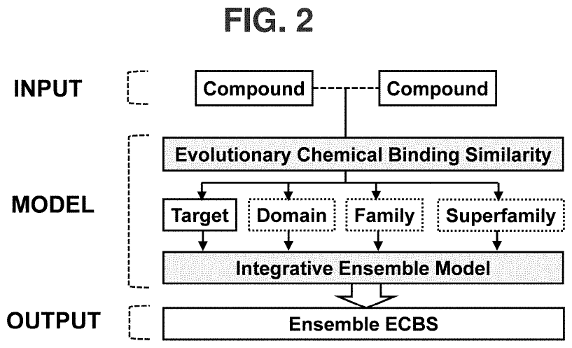

The ensemble classification model combines the advantages of several machine learning models and compensates for their disadvantages, allowing for estimating more accurate binding similarity scores (FIG. 2). Further, meaningful chemical pairs may be searched without a predefined target protein since the chemical binding similarity score contains evolutionary information of targets.

Further, when the positive chemical pairs are restricted to specific targets or targets having evolutionary relationship to the targets (TS-ensECBS model), binding similarity of chemical pairs may be target-specifically defined, and by doing so, higher sensitivity and accuracy may be expected. The chemical binding similarity searching method of the present invention may be referred to as an integrated model considering both of the cases.

The chemical binding similarity searching method using protein evolutionary information of the present invention may quantitate the evolutionary target-binding information as similarity scores for the relationship between chemical compounds, where the similarity scores may compare more complicated binding features between chemical compounds. Accordingly, it is expected that the chemical binding similarity searching method may be widely used with applications such as large-scale ligand-based screening, target-specific ligand identification, drug-repositioning, and general chemical binding similarity calculations by modeling functional similarity.

Hereinafter, the present invention will be described in more detail with reference to Examples. However, these Examples are only for more specifically explaining the present invention, and the scope of the present invention is not intended to be limited by these Examples.

### Example 1. Collection of Binding Information of Chemical and Target Protein

Chemical structures and target protein-binding information were collected from the DrugBank and BindingDB databases. In the DrugBank database, drug-target interaction data (2017 Jul. 28) were retrieved only for “polypeptide” targets and used to obtain SDF (Structure Data Format) files for the drugs. In the BindingDB database, the 2-D SDF file was downloaded (2018 Apr. 1) and parsed to obtain binding affinity data represented by Ki, IC50, Kd, and EC50 values. To exclude low-affinity promiscuous binding, interactions were considered only when the affinity determined by any of the measurements was 100 nM or lower.

As a result, the total numbers of chemicals, target proteins, and binding information were 6671, 4283 and 16587 in DrugBank, and 587693, 5425, and 1018895 in BindingDB, respectively. The two databases were integrated after removing molecules having the same structures by comparing InChiKey.

### Example 2. Collection of Evolutionary Information of Target Proteins

To extract protein sequence-based evolutionary information, domain, family, and superfamily information of the binding target proteins were extracted from various evolutionary information databases including UniprotKB, PFAM, SMART, PRINT, Gene3D, and TIGRFAM. Identifiers for binding targets were unified by UniprotKB entry name. Information of the InterPro database was used to unify different serial numbers from each database, such as UniprotKB-PFAM, UniprotKB-SMART, UniprotKBPRINT, UniprotKB-Gene3D, and UniprotKB-TIGRFAM, by UniprotKB identification numbers, and the protein sequence-based evolutionary information was added to target proteins.

Further, to add protein structure-based evolutionary information, the superfamily database was used. The superfamily server provided hidden Markov models (HMMs) pre-built for 2478 sequenced genomes, which enabled flexible structural protein domain annotation for the target genes using the SCOP family and superfamily ID. The HMM library (http://supfam.org/SUPERFAMILY/downloads/license/supfam-local-1.75/) in the superfamily database was applied to all target sequences using the script “superfamily.pl”.

Through these procedures, overall evolutionary information of target proteins including sequence- and structure-based evolutionary information of target proteins was collected.

### Example 3. Structural Fingerprint Generation for Generating Feature Vectors of Chemical Pairs

Structural information (SDF Lie) for each chemical compound was converted to chemical binary fingerprints using ChemmineR and ChemmineOB cheminformatics packages in R. A fingerprint is a collection of features regarding local fragments found within a chemical structure and is represented by a vector of 0 and 1 values, where 1 and 0 indicate ‘existence’ and ‘absence’ of each feature of a specific chemical structure.

MACCS (256 bits) and FP4 (512 bits) fingerprints available in the ChemmineOB package were concatenated to represent each chemical compound using a 768-bit vector. Further, the fingerprints with empty values for all drugs in DrugBank were discarded to reduce the size of fingerprint vector, which eventually generated a 386-bit feature vector representing an individual chemical compound. The feature vector for a chemical pair was generated by element-wise summation of the chemical fingerprints.

Vij=Vji=Vi+Vj

where Vi is a fingerprint vector for chemical i, and Vj is a fingerprint vector for chemical j.

The element-wise summation of Vi and Vj generated Vij, a feature vector for a chemical pair, where the elements 0, 1, and 2 indicate ‘none’, ‘different’, and ‘common’ features, respectively.

### Example 4. Generation of Data of Negative Chemical Pairs Related to Positive Chemical Pairs

Sampling of negative data is important to determine performance of the machine learning classification model, because the current: chemical-target binding data is highly imbalanced. Thus, a procedure for negative data sampling was designed to balance between the positive sample and the negative sample, and thus, to avoid overfitting problem data.

In detail, six negative chemical pairs for each positive chemical pair were generated. Data of chemical pairs were generated by finding each negative chemical pair which is structurally similar but evolutionarily unrelated to the corresponding positive chemical pair. Specifically, three compounds most structurally similar to Pa and Pb, which constitute a positive chemical pair Pa-Pb were selected. As a result, three molecules (Na1, Na2, and Na3) most similar to Pa were paired with Pb, resulting in three negative chemical pairs of Pb-Na1, Pb-Na2, and Pb-Na3. An identical procedure for Pb generated another three negative chemical pairs of Pb-Na4, Pb-Na5, and Pb-Na6. The generated negative data were excluded if positive chemical pairs were found, followed by repeating the procedure.

### Example 5. Target Binding Similarity Model Through Machine Learning Classification Model

Chemical data of the collected chemical pair and evolutionary information of binding target were used to define classification problem of machine learning, and used to train ECBS models. The model is defined as follows:
Training data: (V11, V12, V13, . . . , Vnm).

Where Vnm is a feature vector for a chemical pair calculated from fingerprint vectors Vn and Vm of an arbitrary chemical pair (n,m).
Data label: {111, 112, 113, . . . , lnm}.

where lnm is a label representing the evolutionary relationship for the chemical pair (n,m), i.e., a target value of the machine learning model.

$${l}_{n m} = \{ \begin{matrix}1 ( positive ) & if Ev ( {V}_{n} ) = Ev ( {V}_{m} ) \\ 0 ( negative ) & otherwise\end{matrix}$$

where EV(Vn) represents evolutionary information for a chemical compound Vn. Accordingly, the positive chemical pairs may be defined in many different ways according to the evolutionary information. For example, in the target information-based ECBS model (Target-ECBS), a chemical pair binding to a common target protein may be defined as a positive sample, whereas in family information-based ECBS model (Family-ECBS), a chemical pair having common family annotation in the binding targets, even though not binding to the same target, may be defined as a positive sample.

Generalizing this, the ECBS model trained with positive chemical pairs defined by evolutionary information “X” (e.g., target, motif, family, or superfamily) is called X-ECBS. In the above formula, the data label is the target value to classify by machine learning, and it suggests that the target value may vary even in the same chemical pairs because each X-ECBS model uses different evolutionary information.

On the other hand, in a target-specific ECBS model (i.e., TS-X-ECBS), only chemical compounds known to bind to a given target or an evolutionarily related protein are collected, and positive chemical pairs are defined therein. This makes it easier to create models by focusing on only chemical compounds related to a specific target when the data size of the chemical compound to be considered is too large. Since the model is created only with information that is evolutionarily related to the specific target, there is an advantage of expecting higher performance when searching for chemical compounds binding to the corresponding target. Similar to the X-ECBS model, TS-X-ECBS model was generated through each evolutionary information “X” (target, Pfam, SMART, PRINT, Gene3D, TIGRFAM, family, or superfamily, etc.) defined for a given target.

### Example 6. Evolutionary Information Integrated Chemical Binding Similarity Model Through Ensemble of Multiple ECBS Models

Application of various machine learning classification models is possible. However, in the present invention, “ranger”, a fast implementation of random forest classifier, was used, because it features adjustable parameters, fast runtime, and efficient memory usage suited for high-dimensional data. For training all the ECBS models, ranger parameters were set with the following options: num.trees=200 or 500, save.memory=TRUE, and down-weighting negative samples by 0.35 with the case.weights option.

A secondary ensemble classifier integrating X-CBS models (i.e., ensECBS model) was built, which is a model calculating common-target binding probability of chemical pairs based on the output scores from the individual X-ECBS models (FIG. 2). This was also generated through the random forest method. An ensemble classifier integrating target-specific TS-X-ECBS models (i.e., TS-ensECBS model) was also built in the same manner, which is a model calculating common-target binding probability of chemical pairs based on the output scores from all TS-X-ECBS models. The difference from the ensECBS model is that information about the amount of data used for training is used as input of the secondary ensemble classifier, along with the output score of the X-ECBS model, to down weight the scores calculated by the TS-X-ECBS models which were built on insufficient amount of evolutionary information data.

The two ensemble classifiers, TS-ensECBS and ensECBS models, were found to be complementary to each other. In other words, TS-ensECBS is suitable for searching for chemical pairs binding to a specific target with high performance, while ensECBS is suitable for predicting the unknown chemical-target interaction. The ensECBS model may also be a useful method of searching for chemical similarity that reveal hidden relationships of chemicals in the absence of direct target-binding data.

### Example 7. Performance Evaluation by Precision-Recall Curve

Area under the curve (AUC) values in precision-recall (PR) were calculated to estimate the prediction performance of each model.

$$Precision = \frac{TP}{TP + FP}$$

$$Recall = \frac{TP}{TP + FN}$$

The higher sensitivity of the PR curve towards positive samples makes it more suitable for the evaluation of model performance by focusing on positive samples. The R package ‘PRROC’ was used to calculate AUC values.

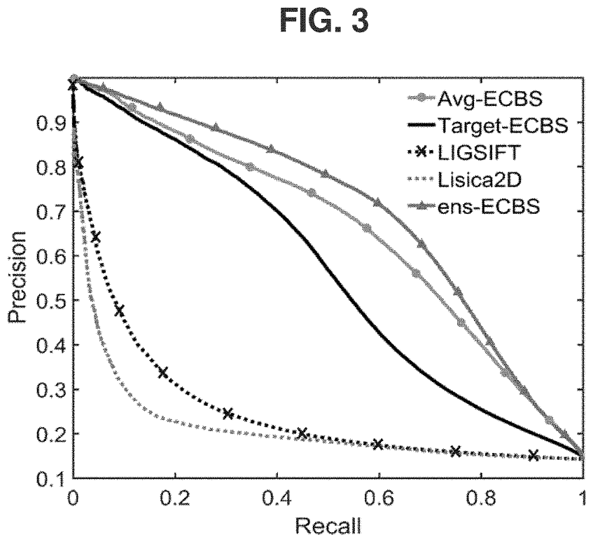

As a result of testing performance using the AUC values of PR curve, ensECBS showed more excellent performance than Target-ECBS, suggesting that evolutionary information was effective to improve the prediction performance. The secondary ensemble procedure in ensECBS was more effective than just averaging all individual X-ECBS scores (Avg-ECBS). Further, ensECBS also showed higher performance than the existing structure similarity methods (LIGSIFT, Lisica2D) (FIG. 3).

This suggests that the classification model constructed by heterogeneous target binding chemical pairs with various evolutionary information is effective for correctly predicting evolutionarily related compounds without direct chemical-target binding information.

### Example 8. Prediction of Chemical Pairs Binding to Ephrin Type-B Receptor 4

To examine chemical pair prediction accuracy of the ensECBS which is the chemical binding similarity searching method using protein evolutionary information of the present invention, the 2D structure similarity method and the ensECBS of the present invention were compared with each other by examining prediction accuracy for the chemical pairs binding to Ephrin type-B receptor 4.

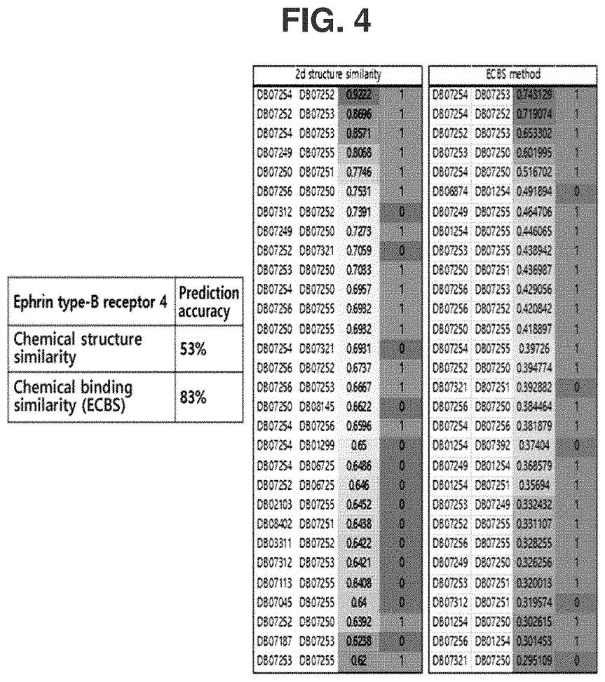

As a result, the top-scored 30 data of drug pairs were constructed by each method. When the top-scored drug pairs were sorted by each similarity score, it was confirmed that the existing 2D structure similarity method showed accuracy of 53% whereas the ensECBS method of the present invention showed prediction accuracy of 83% (FIG. 4). Tanimoto coefficient based on chemical fingerprint was used for representing 2D structure similarity whereas ensECBS method was used for the evolutionary chemical binding similarity.

Taken together, since the chemical binding similarity searching method using protein evolutionary information of the present invention includes protein-binding information of chemicals, it may be a useful technique to search for chemical compounds with similar functions and to reveal their mechanism of action. Further, the method may be widely used in chemical binding similarity calculations such as large-scale ligand-based screening, target-specific ligand identification, drug-repositioning, etc.

Based on the above description, it will be understood by those skilled in the art that the present invention may be implemented in a different specific form without changing the technical spirit or essential characteristics thereof. Therefore, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

### Effect of the Invention

A chemical binding similarity searching method using protein evolutionary information of the present invention may be widely applied for general-purpose, and is a chemical similarity searching method capable of representing not structure similarity but target-binding similarity, which may be a useful technique to search for chemical compounds having similar functions with higher sensitivity and to reveal their mechanism of action.

Further, the evolutionary target binding information is quantitated as a similarity score for representing the relationship between chemical compounds, where the similarity scores may compare more complicated binding features between chemical compounds. Accordingly, it is expected that the method may be widely used with applications such as large-scale ligand-based screening, target-specific ligand identification, drug-repositioning, and general chemical binding similarity calculations by modeling functional similarity.

What is claimed is:

1. A chemical binding similarity searching method using protein evolutionary information, the method comprising the steps of:
obtaining chemical-target protein binding information from experimental data;
constructing expanded chemical-protein interaction data by using diverse evolutionary information of the target proteins;
categorizing the interaction data into positive and negative chemical pairs and quantitating the data; and
applying a machine learning-based classification model to the quantitated data to calculate a chemical binding similarity score.

2. The chemical binding similarity searching method using protein evolutionary information of claim 1, wherein the database of binding information includes DrugBank or BindingDB.

3. The chemical binding similarity searching method using protein evolutionary information of claim 1, wherein the evolutionary information of the target protein is motif, domain, family, or superfamily.

4. The chemical binding similarity searching method using protein evolutionary information of claim 1, wherein the chemical pairs are numerically represented by using structural fingerprints of the chemicals.

5. The chemical binding similarity searching method using protein evolutionary information of claim 4, wherein the structural fingerprints of the chemical pairs use the following equation:
Vij=Vji=Vi+Vj
(V is a fingerprint vector, Vi is a fingerprint for chemical i, and Vj is a fingerprint for chemical j).

6. The chemical binding similarity searching method using protein evolutionary information of claim 1, wherein the positive chemical pair is a chemical pair binding to a common target protein or a chemical pair binding to a target protein having common evolutionary information.

7. The chemical binding similarity searching method using protein evolutionary information of claim 1, wherein the negative chemical pair is structurally similar to the positive chemical pair but evolutionarily unrelated to the binding target protein.

8. The chemical binding similarity searching method using protein evolutionary information of claim 1, wherein the multiple machine learning classification models defined by different evolutionary target information is integrated to build the secondary classification model.

9. The chemical binding similarity searching method using protein evolutionary information of claim 6, wherein the machine learning classification model includes naive bayes classifier, support vector machine, random forest, neural network, or deep learning.
